(19) United States
(12) Patent Application Publication
(10) Pub. No.: US 2005/0214356 A1
(43) Pub. Date: Sep. 29, 2005

(54) Self assembling activation agents targeted using active drug release

(76) Inventor: Timothy H. Joyce, Mountain View, CA (US)

Correspondence Address: Joel N. Bock, Esq.;SILLS CUMMIS EPSTEIN & GROSS P.C., One Riverfront Plaza, Newark, NJ, 07102-5400, US

(21) Appl. No.: 10/807,835

(22) Filed: Mar. 24, 2004

(51) Int. Cl. A61K 38/12; A61K 9/127; A61K 9/14

(52) U.S. Cl. 424/450; 514/9

(57) ABSTRACT

In a method for therapeutic treatment of humans and other mammals, the therapeutic composition includes a fusogenic encapsulation vesicle and an activation agent such as an organic nanotube or α-DL peptide enclosed in the encapsulation vesicle. The activation agent is maintained in an inactivated form and becomes activated by an activation condition.

### CROSS REFERENCE TO RELATED APPLICATIONS

The present application is a continuation-in-part of a currently pending application Ser. No. ______, which was filed on Jan. 30, 2004, which is a continuation-in-part of a currently pending application Ser. No. 10/465,407, which was filed on Jun. 18, 2003, which is a continuation-in-part of a currently pending application Ser. No. 10/387,301, which was filed on Mar. 11, 2003. All of these applications are specifically incorporated herein by reference.

### FIELD OF THE INVENTION

The present invention is in the field of encapsulation vesicles that contain self assembling activation agents such as pore forming agents, cyclic peptides or nanotubes that may be enclosed in encapsulation vesicles and targeted for treatment of disease.

### BACKGROUND OF THE INVENTION

Liposomes are small vesicular sacs that resemble tiny cells. These sacs have an aqueous or hydrophilic interior volume separated generally by a durable hydrophobic bilayer membrane. Both water-soluble drugs and insoluble drugs can, therefore, be incorporated into these vesicles. Depending upon the production process used, these vesicles may comprise a single membrane (unilamellar) or several membranes (multilamellar). This makes construction of such vesicles quite flexible. In addition, the typical size of these liposomes can be selected to range from 0.05 to several micrometers in diameter. The ability to design vesicles of varying size makes these vehicles an effective delivery agent for a variety of cellular targets.

Since their discovery, more than 35 years ago, liposomes have been used in a variety of ways to deliver a variety of different drugs. The prospect of targeting liposomes to cancer or tumor sites generated a considerable excitement in medical research in the 1960's and 1970's. Early liposome formulations, however, were no more effective than non-encapsulated formulations. For instance, studies of administering doxorubicin encapsulated liposomes showed little improved antitumor activity. The lack of improved antitumor activity was largely due to the fact that liposomes were unstable in blood and released a good portion of their drug contents as a consequence of rapid binding of plasma proteins (opsonization). Exposure of liposomal preparations to normal plasma proteins showed evidence of hundreds of bands of these proteins called “opsins” bound to the outer surface of the liposome. These proteins would then act to mark the liposome as a foreign body for removal from the blood stream by the body's natural defense mechanisms. Secondly, liposomes that survived the destabilization process were immediately sequestered by the fixed macrophages in the spleen and liver (the mononuclear phagocyte system (MPS)). Once internalized by macrophages, the liposomes were destroyed. The combination of instability and rapid uptake by the MPS system severely limited the use of liposomes as drug delivery systems (DOXIL Clinical Series Vol. 1, No. 1, 1997).

Researchers realized that something needed to be done to improve liposome circulation times as well as to lower detection by the body's immune defenses. Comparisons were made to erythrocytes (red blood cells) and other similar cells that circulate for extended periods of time before degradation by macrophages. It was determined that red blood cells have a thick coat of carbohydrate on their surface and this allows them to circulate for extended periods of time (Allen, T. M., Chonn, A. “Large Unilamellar liposomes with low uptake by the reticuloendothelial system”. FEBS Lett. 1987; 223:42-46). As a result, researchers were able to graft MPEG (methoxypolyethylene glycol), a hydrophilic polymer, onto the surface of the liposomes. These new liposomes were found to have extended longevity in plasma (Papahadjopouos, D., Allen, T. M., Gabizon, A., Barenholz, Y., “Optimization and Upscaling of Doxorubicin-containing liposomes for clinical use. Journal of Pharmaceutical Sciences Vol. 79, No. 12, December 1990). One of the most effective liposomes called “Stealth® liposomes” were shown to stably encapsulate doxorubicin, re-circulate for periods of several days after injection without releasing drug, penetrate into tumor cells, and release encapsulated drug within the tumor. The long residence times of the Stealth® liposomes may be explained by the steric stabilization effect provided by the MPEG molecules on the surface of the vesicles. In other words, the liposome surface comprises a protective hydrophilic layer that prevents interaction of the plasma components with the liposomes. As a result, the Stealth® liposomes may circulate longer in the blood stream.

Even today, the mechanism of why these liposomes work effectively remains unclear. However, the actions within tumors may help explain. For instance, the liposome is quite small (the average is approximately 100 nm) and this allows for optimized drug carrying and circulation time. In addition, most solid tumors exhibit unique pathoanatomic features, such as extensive angiogenesis, hyperpermeable and defective architecture, impaired lymphatic drainage, and greatly increased production of mediators that enhance vascular permeability. These conditions allow for Stealth® liposomes to extravasate in solid tumors through defects present in the endothelial barriers of newly forming blood vessels. Consistent with this observation, in addition, inflammatory tissue and tissues with local infections also contain vasculature with greatly enhanced permeability and, therefore, have been shown to be targets for efficient Stealth® liposome extravasation. Extravasation of these and other type liposomes probably occur between gaps and other similar spaces that allow the liposomes to lodge themselves between tumor cells.

Once there, it is believed that the enclosed drug material is released either by leakage or by liposome degradation caused by enzymes such as phospholipases (Working, P. K., Newman, M. S., Huang, S. K., et al. “Pharmacokinetics, biodistribution and therapeutic efficacy of doxorubicin encapsulated in Stealth® liposomes (DOXIL®). Liposomes Res. 1994; 4:667-687). Although there is no solid evidence, it is postulated that the release of drugs into tumor cells probably occurs over a period of days and possibly weeks. If this hypothesis holds true, then the tumors would be exposed to high concentrations of drugs for prolonged periods of time, thus enhancing the efficacy of the chemotherapeutic effects.

However, these systems suffer from a few important limitations. First of all, the actual dosage levels and delivery of drug concentrations remains unclear. Without knowledge of the delivery mechanism or dosage effect, treatments remain fairly crude on a “hit or miss” approach. In addition, degradation is likely to be incomplete in some vesicles, or not at all. Therefore, the overall efficiency and delivery regulation could be improved. In addition, exposure of tumor tissue to drugs over time often results in resistance of the tumor to the drugs. This occurs through exposure and rapid mutation of tumor cells during the stages of metastasis. Therefore, there is a strong need to provide a more precise method for high dosing of drug in targeted areas at prescribed time intervals to avoid potential drug resistance problems. There is also the need to be able to more precisely and predictably control the delivery of drugs or to deliver the drugs more quickly to defined locations to minimize overall complications, development of drug resistant cells and to lower chemotherapeutic side effects in the patient. Furthermore, a need always exists to develop new agents to be used in combination with liposomes to take advantage of their unique properties while enhancing their performance in specific clinical indications.

While liposomes offer one approach to selectable, targeted drug delivery, other techniques and compositions have been developed to attempt to increase the local concentration of an active agent. Liposomes provide this function by encapsulating an active agent that typically does not change its active characteristics between the point of encapsulation and the point of delivery.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989)). When liposomes are endocytosed by a target cell, for example, they can be routed to acidic endosomes which will destabilize the liposome and result in drug release. Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release. In addition to the foregoing methods, a liposome having a predetermined phase transition temperature, T_{C}, above body temperature can be used to achieve active drug delivery. In this method, the body temperature will maintain the liposome below the T_{C} so that the liposome will not become leaky when placed in the body. This method of drug release is capable of “on demand” drug delivery since such liposomes experience a greatly increased membrane permeability at their T_{C} which, in turn, enables drug or chemical release. To release drugs from such phase transition liposomes when in the body, heat must be applied until the T_{C} is achieved. Unfortunately, the application of heat can, in itself, create problems within the body and, frequently, the adverse effects of the heat treatment outweigh the beneficial effects of using the liposome as a drug delivery vehicle. Moreover, such liposomes must be made of highly purified and expensive phase transition phosopholipid materials.

In view of the foregoing, a number of novel systems were developed that use active targeting for drug delivery that overcomes the disadvantages of the above described methods. Specifically, parenteral delivery systems have been developed that are stable in circulation, following intravenous administration, allowing retention of encapsulated or associated drug or therapeutic agent(s). These delivery systems are capable of accumulating at a target organ, tissue or cell via either active targeting (e.g., by incorporating an antibody or hormone on the surface of the liposomal vehicle) or via passive targeting, as seen for long-circulating liposomes. Following accumulation at the target site, the liposomal carrier becomes fusogenic, without the need for any external stimulus, and subsequently releases any encapsulated or associated drug or therapeutic agent in the vicinity of the target cell, or fuses with the target cell plasma membrane introducing the drug or therapeutic agent into the cell cytoplasm. To date, however, such delivery systems have only been successful in delivery nucleic acids, plasmids or drugs. Delivery of peptides or other agents have been unsuccessful due to their toxicity, lack of chemical flexibility, instability, or inability to be controlled or activated upon demand. Other compositions rely on the ability to be administered without encapsulation, but to change activity upon exposure to a certain condition. These compounds, termed activation agents, change their biochemical characteristics in vivo.

In contrast, non-targeted activation agents such as organic nanotubes are being used and developed to treat bacterial, viral and other diseases. Certain activation agents such as pore forming agents are being developed to address drug resistance problems in bacteria and other microorganisms. In addition, limited toxicity studies in mice have shown that some of these self-assembling materials may be effective for in vivo application (Bong, D. T, et al., “Self-Assembling Organic Nanotubes, Angew. Chem. Int. Ed. 2001, 40, 988-1011). These activation agents, can be easily synthesized, are flexible in design and can quickly self-assemble. However, non-targeted activation agents have limited utility in therapeutic applications. First of all, the activation agents can not be delivered site specifically and remain questionable regarding overall efficacy, and ability to self-assemble in vivo. Secondly, the naked dosing of activation agents to patients is likely to cause severe immunological or toxicological problems. Lastly, targeting of these components can be a problem.

Therefore, there is a strong need to provide a therapeutic composition or method for delivering these activation agents site specifically in vivo with limited toxicological or immunological response. In addition, being able to deliver these compounds to tumors and cancer cells and then to activate them to destroy these cells is also desired. The present invention satisfies these and other described needs.

### SUMMARY OF THE INVENTION

The present invention relates to a composition of matter for therapeutic treatment of humans and other mammals. Basically, the therapeutic composition of the present invention comprises an encapsulation vesicle such as a liposome, and an active or inactive activation agent such as an organic nanotube/cyclic peptide enclosed in the encapsulation vesicle. Each of these components has special requirements to be effective in human therapeutic and diagnostic uses in vivo.

The encapsulation vesicle must be large enough to allow an activation agent such as an organic nanotube within its interior. The encapsulation vesicle may optionally allow the attachment of a targeting ligand and/or enclose a bioactive agent. In certain instances the encapsulation vesicle may be smaller than 600 nm in diameter. In other instances, the encapsulation vesicle may be from about 20 nm to about 100 nm, or from 100 nm to about 600 nm, but no less than 10 nm. In most cases, the liposomes are most effectively constructed at about 100 nm. This will allow for maximum extravasation of the vesicles into cancer tumors and other sites of infection. Preferential localization of long-circulating liposomes at sites of infection and inflammation has been demonstrated in a variety of experimental models and human clinical trials. Anti-infective agents encapsulated in liposomes provide improved therapy, relative to that provided by unencapsulated drugs. In animal models, the extent of liposome localization at sites of infection demonstrated a positive linear correlation to the area under the blood concentration time curve (AUC) of the liposome formulations, once again highlighting the positive effect of the mPEG coating. Gamma scintigraphy has been used to successfully image sites of inflammation following injection of long-circulating liposomes containing technetium-99m in animal models and human patients. Most solid tumors exhibit unique pathoanatomic features, such as extensive angiogenesis, hyperpermeable and defective vasculature architecture, impaired lymphatic drainage, and greatly increased production of mediators that enhance vascular permeability. Liposomes extravasate in solid tumors through gaps present in the normally continuous vascular endothelium. The size of these gaps has been measured in a variety of implanted tumors in mice and found to be no larger than about 0.6 microns. This size represents upper bounds for gaining access to tumor tissue. In agreement with this hypothesis, the rate and extent of extravasation in solid tumors is critically related to liposome size (and plasma residence time). Particles with an average diameter greater than 600 nm do not extravasate at all. For particles with diameters below 600 nm, the rate of extravasation appears to increase with smaller diameters. An important component of the invention is the activation agent. The activation agent may be optionally activated by an activation condition. For instance, if the activation agent is an organic nanotube, it may be designed to activate by internal or external activation conditions that allow the nanotube to self-assemble into supramolecular structures in the membrane of a particular type of pathological cell such as a cancer cell.

The invention also provides a method for therapeutic treatment using the composition of the invention. The composition is formulated for parenteral administration (i.e. by injection, for example, bolus injection or continuous infusion) to the patient where it is taken up or extravasated by the necrotic or infected tissues. The encapsulation vesicle is designed so that the activation agent remains in the encapsulation vesicle and is delivered to a desired tissue or cell.

The method for therapeutic treatment may also comprise contacting a cell membrane with a therapeutic composition that comprises an encapsulation vesicle and an active or inactive activation agent such as an organic nanotube enclosed in the encapsulation vesicle and allowing the cell membrane to incorporate the therapeutic composition so that the activation agent of the therapeutic composition may incorporate into the particular type of cell or be activated to incorporate in the particular type of cell membrane.

In another embodiment, the present invention provides a fusogenic liposome comprising a lipid capable of adopting a non-lamellar phase, yet capable of assuming a bilayer structure in the presence of a bilayer stabilizing component; and a bilayer stabilizing component reversibly associated with the lipid to stabilize the lipid in a bilayer structure. Such fusogenic liposomes are extremely advantageous because the rate at which they become fusogenic can be not only predetermined, but varied as required over a time scale ranging from minutes to days. Control of liposome fusion can be achieved by modulating the chemical stability and/or exchangeability of the bilayer stabilizing component(s). By controlling the composition and concentration of the bilayer stabilizing component, one can control the chemical stability of the bilayer stabilizing component and/or the rate at which the bilayer stabilizing component exchanges out of the liposome and, in turn, the rate at which the liposome becomes fusogenic. In addition, other variables including, for example, pH, temperature, ionic strength, light or irradiation by UV, IR, microwave, heat or some other physical or chemical agent can be used to vary and/or control the rate at which the liposome becomes fusogenic.

As described in EPO Patent No. EPO 723554 B1, the kinetics of assembly and disassembly of cyclic peptide to form molecular tubes can be controlled by the selection of amino acid side chain groups. Cyclic peptides with ionizable amino acid side chains display pH dependent kinetics. Charged cyclic peptides are found to resist tube assembly; neutralized cyclic peptides are found to promote tube assembly. For example, cyclic peptides incorporating glutamic acid are found to spontaneously assemble into molecular tubes at acidic pH but resist assembly into molecular tubes at alkaline pH. Pre-assembled molecular tubes are found to spontaneously disassemble when the pH is raised from acid pH to alkaline pH. Judicious selection of amino acid side chains can promote packing or aggregation of molecular tubes to form tubular bundles.

In another embodiment, the present invention provides a method for delivering a therapeutic compound to a target cell at a predetermined rate, the method comprises administering to a host containing the target cell a fusogenic liposome which comprises a bilayer stabilizing component, a lipid capable of adopting a non-lamellar phase, yet capable of assuming a bilayer structure in the presence of the bilayer stabilizing component, and a therapeutic compound such as an organic nanotube or cyclic peptide with a pharmaceutically acceptable salt thereof. Administration may be by a variety of routes, but the therapeutic compounds are preferably given intravenously or parenterally. The fusogenic liposomes administered to the host may be unilamellar, having a mean diameter of 0.05 to 0.45 microns, more preferably from 0.05 to 0.2 microns.

In yet another embodiment, the present invention provides a cyclic lipopeptide, the cyclic lipopeptide comprises a lipid covalently attached to a cyclic peptide by means of an amide bond. Typically, the amide bond is formed between a carboxyl group of the lipid and an amino group of the peptide. In addition, the present invention provides a pharmaceutical composition for introducing a cyclic peptide or organic nanotube into a cell of a host, the pharmaceutical composition comprising a liposome containing a lipopeptide, the lipopeptide comprising a lipid covalently attached to a peptide by means of an amide bond; an organic nanotube or cyclic peptide enclosed in the liposome; and a pharmaceutically acceptable carrier. Such liposomes are stable at physiological pH, but after being internalized by cells through an endocytic pathway, the liposomes exposed to the acidic pH of the endosome are destabilized and fuse with the endosome membrane, resulting in release of their contents into the cytoplasm. Remarkably, the activation agents such as the cyclic peptide or nanotubes can be concomitantly activated by pH change to form supramolecular structures in membranes of nearby cancer cells. These supramolecular structures form pores in the cells and destroy them. In addition, in dense tissues of infection, necrosis or tumors, such pores may prove beneficial for subsequent drug treatments should the initial drugs prove ineffective in irradicating the cancer or tumor.

### BRIEF DESCRIPTION OF THE DRAWINGS

FIG. 1 shows a schematic representation of an encapsulation vesicle enclosing an activation agent of the present invention;

FIG. 2 shows how the encapsulation vesicle may be used to deliver and actively target an activation agent to a tissue or tumor;

FIG. 3 shows how the activation agent is taken up, activated and released into a particular type of cell such as a cancer cell.

### DESCRIPTION OF THE SPECIFIC EMBODIMENTS

Compositions and methods for therapeutic treatment are provided. In the described methods the therapeutic composition may be used to contact a cell membrane. The cell membranes may be in vitro or in vivo and include both pathogenic and nonpathogenic cells unless clearly stipulated otherwise.

Before describing the invention in further detail, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a”, “an” and “the” include plural reference unless the context clearly dictates otherwise.

“Activate” or “activate by an activation condition” refers to the application of physical, chemical or biochemical conditions or processes that will cause an activation agent to assemble into a supramolecular structure or self assemble in a cell membrane.

“Active” refers to the ability to self assemble into supramolecular structures without any additional physical, chemical or structural modification of the activation agent.

“Inactive” refers to the immediate inability to self assemble into a supramolecular structure, but capable of becoming active and activated by an activation condition.

“Carrier” refers to a pharmaceutically acceptable vehicle, which is a nonpolar, hydrophobic solvent, and which may serve as a reconstituting medium. The carrier may be aqueous based or organic based. Carriers include, inter alia, lipids, proteins, polysaccharides, sugars, polymers, copolymers, and acrylates.

“Cell” refers to any one of the minute protoplasmic masses that make up organized tissue, comprising a mass of protoplasm surrounded by a membrane, including nucleated and unnucleated cells and organelles.

“Cell membrane” refers to the commonly described lipid-based exterior boundary of a cell. The cell membrane may or may not comprise proteins or receptors.

“Diseased cell”, “pathogenic cell” or “pathological cell” refers to any cell that fails to operate in its naturally occurring condition or normal biochemical fashion. These cells should be capable of causing disease. For instance, the word shall include cells that are subject to uncontrolled growth, cellular mutation, metastasis or infection. The term shall also include cells that have been infected by a foreign virus or viral particle, bacteria, bacterial exotoxins or endotoxins, prions, or other similar type living or non-living materials. The term may in particularly refer to cancer cells or cells infected by the polio virus, rhinovirus, piconavirus, influenza virus, or a retrovirus such as the human immunodeficiency virus (HIV).

“Fusion” refers to the joining together of components to form a single contiguous component. For instance, when two cell membranes contact each other the lipids, proteins or other cellular materials re-associate and/or reorganize to form a single contiguous membrane.

“Lipid” refers to a naturally occurring, synthetic or semi-synthetic (i.e. modified natural) compound that is generally amphipathic. The lipids typically comprise a hydrophilic component and a hydrophobic component. Exemplary lipids include, for example, fatty acids, neutral fats, phosphatides, oils, glycolipids, surface active agents (surfactants), aliphatic alcohols, waxes, terpenes and steroids. The phrase semi-synthetic (or modified natural) denotes a natural compound that has been chemically modified in some fashion.

“Liposome” refers to a generally spherical or spheroidal cluster or aggregate of amphipathic compounds, including lipid compounds, typically in the form of one or more concentric layers, for example bilayers. They may also be referred to as lipid vesicles. The liposome may be formulated, for example, from ionic lipids and/or non-ionic lipids. Liposomes formulated from non-ionic lipids may be referred to as niosomes.

“Nanocomposites” refers to composite structures whose characteristic dimensions are found on the nanoscale. An example is the suspension of carbon nanotubes in a soft plastic host.

“Nanodot” refers to nanoparticles that consist of homogenous material, especially those that are almost spherical or cubical in shape.

“Nanoparticle” refers to any material that can be made, ground or produced on the nanoscale.

“Nanorod” refers to nanostructures that are shaped like long sticks or dowels, with a diameter in the nanoscale and a length not very much longer.

“Nanoscale” refers to phenomena that occur on the length scale between 1 and 100 nanometers.

“Nanostructure” refers to structures whose characteristic variation in design length is on the nanoscale.

“Nanowire” refers to nanorods that can conduct electricity.

“Patient” refers to animals, including mammals, preferably humans.

“Polymer” refers to molecules formed from chemical union of two or more repeating units. Accordingly, included within the term “polymer” may be, for example, dimers, trimers and oligomers. The polymer may be synthetic, naturally occurring or semi-synthetic. The term may refer to molecules that comprise 10 or more repeating units.

“Protein” refers to molecules comprising essentially alpha-amino acids in peptide linkages. Included within the term “protein” are globular proteins such as albumins, globulins and histones, fibrous proteins such as collagens, elastins and keratins. Also included within the term are compound proteins, wherein a protein molecule is united with a non-protein molecule, such as nucleoproteins, mucoproteins, lipoproteins and metalloproteins. The proteins may be naturally occurring, synthetic or semi-synthetic.

“Receptor” refers to a molecular structure within a cell or on the surface of a cell that is generally characterized by the selective binding of a specific substance. Exemplary receptors include cell surface receptors for peptide hormones, neurotransmitters, antigens, complement fragments, immunoglobulins and cytoplasmic receptors for steroid hormones. Receptors may also comprise intracellular receptors such as those on the surface of the nuclear membrane (i.e. PPARs).

“Region of a patient” refers to a particular area or portion of the patient and in some instances to regions throughout the entire patient. Examples of such regions include the eye, gastrointestinal regions, cardiovascular regions (including myocardial tissue), circulatory system, bladder, mucosa, renal region, vascular tissues, as well as disease tissue such as cancerous tissue including prostate, breast, gallbladder, and liver. The term includes, for example, areas to be targeted by a drug delivery device or a bioactive agent. The term refers to both topical and internal organs and tissues. The phrase “vascular” or “vasculature” denotes blood vessels (including arteries, veins, and the like). The phrase “gastrointestinal region” includes the region defined by the esophagus, stomach, small intestine, large intestine, and rectum. The phrase “renal region” denotes the region defined by the kidney and the vasculature that leads directly to and from the kidney and includes the abdominal aorta.

“Region to be targeted” or “targeted region” refers to a region where delivery of a therapeutic is desired.

“Solid-state” or “solid state material” refers to materials that are not biological, biologically based or biological in origin. Such materials may include carbon based materials, synthetic fibers, polymers, plastics, semiconductor materials, silica or silicon based substrates or materials, carbon based nanotubes, quantum dots, artificial bone cylinders, magnetic nanoparticles, nanocrystals, suicide inhibitors, nanodots, nanotubes, nanostructures, or nanowires. These structures may be enclosed within, inserted into, comprise a portion of or be attached to the encapsulation vesicles or activation agents. In certain instances they may also comprise the activation agent. These materials should be capable of activation by an activation condition.

“Supramolecular structures” are multi-subunit structures, e.g. nanotubes, barrels and carpets of nanotubules, which are believed to be formed through “noncovalent” assembly. These structures are thermodynamically controlled assemblies that can undergo reversible structural assembly and disassembly. This process will depend upon the environment, subunit structure, side group selection, side group interaction, and the nature and combination of noncovalent forces acting on the system. Hence, an important and attractive feature of these structures is their ability to select amongst various cell membrane types.

“Targeting ligand” or “target ligand” refers to any material or substance that may promote targeting of tissues and/or receptors in vivo or in vitro with the therapeutic compositions of the present invention. The targeting ligand may be synthetic, semi-synthetic, or naturally occurring. Materials or substances which may serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, vitamins, steriods, steriod analogs, hormones, cofactors, bioactive agents, genetic material, including nucleotides, nucleosides, nucleotide acid constructs and polynucleotides.

“Therapeutic” refers to any pharmaceutical, drug or prophylactic agent which may be used in the treatment (including the prevention, diagnosis, alleviation, or cure) of a malady, affliction, disease or injury to a patient. Therapeutic includes contrast agents and dyes for visualization, therapeutically useful peptides, polypeptides and polynucleotides may be included within the meaning of the term pharmaceutical or drug.

“Tissue” refers generally to specialized cells that may perform a particular function. The term refers to an individual cell or plurality or aggregate of cells, for example, membranes, blood or organs. The term also includes reference to an abnormal cell or plurality of abnormal cells. Exemplary tissues include myocardial tissue, including myocardial cells, membranous tissues, including endothelium and epithelium, laminae, connective tissue, including interstitial tissue, and tumors.

“Vesicle” or “encapsulation vesicle” refers to an entity that is generally characterized by the presence of one or more walls or membranes that form one or more internal voids. Vesicles may be formulated, for example, from a stabilizing material such as a lipid, including the various lipids described herein, a proteinaceous material, including the various proteins described herein, and a polymeric material, including the various polymeric materials described herein. As discussed herein, vesicles may also be formulated from carbohydrates, surfactants, and other stabilizing materials, as desired. The lipids, proteins, polymers and/or other vesicle forming stabilizing materials, may be natural, synthetic or semi-synthetic. Preferred vesicles are those which comprise walls or membranes formulated from lipids. The walls or membranes may be concentric or otherwise. The stabilizing compounds may be in the form of one or more monolayers or bilayers. In the case of more than one monolayer or bilayer, the monolayers or bilayers may be concentric. Stabilizing compounds may be used to form a unilamellar vesicle (comprised of one monolayer or bilayer), an oligolamellar vesicle (comprised of more than about three monolayers or bilayers). The walls or membranes of vesicles may be substantially solid (uniform), or referred to as, for example, liposomes, lipospheres, nanoliposomes, particles, micelles, bubbles, microbubbles, microspheres, nanospheres, nanostructures, microballoons, microcapsules, aerogels, clathrate bound vesicles, hexagonal/cubic/hexagonal II phase structures, and the like. The internal void of the vesicle may be filled with a wide variety of materials including, for example, water, oil, gases, gaseous precursors, liquids, fluorinated compounds or liquids, liquid-perfluorocarbons, liquid perfluoroethers, therapeutics, bioactive agents, if desired, and/or other materials. The vesicles may also comprise a targeting ligand if desired.

“Vesicle stability” refers to the ability of vesicles to retain the gas, gaseous precursor and/or other bioactive agents entrapped therein after being exposed, for about one minute, to a pressure of about 100 millimeters (mm) of mercury (Hg). Vesicle stability is measure in percent (%), this being the fraction of the amount of gas which is originally trapped in the vesicle and which is retained after release of the pressure. Vesicle stability also includes “vesicle resilience” which is the ability of a vesicle to return to its original size after release of the pressure.

Where a range of values is provided, it is understood that each intervening value, to the tenth of a unit of the lower limit unless the context clearly dictates otherwise, between the upper and the lower limit of that range, and any other stated or intervening value in that stated range, is encompassed within the invention. The upper and lower limits of these smaller ranges are also encompassed within the invention, subject to any specifically excluded limit in the stated range. Where the stated range includes one or both of the limits; ranges including either or both of those included limits are also included in the invention.

Unless defined otherwise, all technical and scientific terms used herein have the same meaning as commonly understood to one of ordinary skill in the art to which this invention belongs. If for some reasons the usage or definitions herein shall be interpreted to differ from the commonly understand usage, then the definitions, herein, shall prevail. Although any methods, devices and materials similar or equivalent to those described herein can be used in the practice or testing of the invention, the methods, devices and materials are now described. Methods recited herein may be carried out in any order of the recited events that are logically possible, as well as the recited order of events.

All patents and other references cited in this application infra and supra, are hereby incorporated by reference except as they may conflict with those of the present application (in which case the present application prevails).

In further describing the present invention, the therapeutic composition and methods of making the composition are first described in general detail. Then a few representative applications are provided. Subsequently, the method of therapeutic treatment using the therapeutic composition is then described and examples provided.

Activation Agents:

An important component of the invention is the activation agent. The activation agent has a number of important properties. Activation agents can be in active or inactive form. In the active form the activation agents must be capable of forming higher order structures or self assembling into supramolecular structures. In the inactive or inert form the activation agent must be capable of being activated by an activation condition to become active and form supramolecular structures, change conformation, change energy state, or be altered by a chemical, physical or electrical property. The activation agent in its active form must also be capable of destroying or disrupting the cellular biochemistry of the cell or cell membrane it is in or becomes incorporated into. Activation agents may be capable of being transferred or incorporated into the cell membranes or cellular interior of other cells. The activation agent may also be capable of being used as a transmembrane channel to regulate or deliver a drug. The activation agent may be designed to open and close by an activation condition. They may also be activated to assemble, disassemble, organize, form supramolecular structures or incorporate into membranes after being activated by an activation condition. The activation agents may for instance comprise a pore forming agent, a solid state material, a nanotube, a nanorod, a carbon nanotube, a nanocomposite, a nanowire, an ionophore, a nanodot, a quantum dot, a nanostructure, a polymer, a synthetic material, silica or silicon materials, artificial bone or bone material, suicide inhibitors and other similar materials known and previously described in the art. For instance, the activation agent may comprise a nanowire positioned in the encapsulation vesicle that may be activated by an exogenous or endogenous source. The nanowire may become incorporated into a cancer cell (by endocytosis, fusion, or phagocytosis) and then may be irradiated by an external light source to “burn out” the tumor. The activation agents may also comprise a pore forming agent. The pore forming agents may be in inactive form and then are activated by an activation condition. The pore forming agents then self assemble on nearby membranes to form supramolecular structures that disrupt the cellular biochemistry or polarity of the cell that they incorporate into. The invention should not be interpreted to be limited to the above described embodiments and materials and includes other embodiments and materials that maintain the above described properties that are know in the art or that may be developed.

Pore Forming Agents:

One type of activation agent may be a pore forming agent. Active pore forming agents must be capable of self-assembling in cell membranes or walls. They generally comprise monomeric units that aggregate together to form more highly ordered or organized supramolecular structures (i.e. heptamer, hexamer, nantubes, sheets, carpets, etc.). The structures and assembly process may comprise various stages of assembly. The pore forming agents generally work synergistically to form channels that span the cell membrane or wall. The monomeric units may disrupt, but in many conditions do not form the pores individually. These agents may be organic or synthetic in nature. In inactive form pore forming agents will not readily associate or aggregate to form highly ordered or organized supramolecular structures.

The active pore forming agent may have lytic activity and the structures it forms may be capable of being activated to open, close or both. It also may be capable of releasing chemicals or molecules that may prove toxic to a pathogenic cell. Active pore forming agents may be capable of self assembling. Active or inactive pore forming agents may be designed to hold bioactive agents or degrade to release bioactive agents or other materials that may be potentially toxic to a pathogenic cell upon activation by an activation condition.

Pore forming agents may be produced synthetically (i.e. a peptide, peptide fragment, organic nanotube etc.). The pore forming agent can be a molecule or fragment, derivative or analog of such molecules. The pore forming agents may be capable of making one or more lesions or pores in the encapsulation vesicle(s). These pore forming agents may be derived from a variety of bacteria including α-hemolysin, E. coli hemolysin, E. coli colicin, B. thuringensis toxin, aerolysin, perfringolysin, pneumolysin, streptolysin O, and listeriolysin. Eucaryotic pore forming agents capable of lysing cells include defensin, magainin, complement, gramicidin, mellitin, perforin, yeast killer toxin and histolysin. Synthetic organic molecules that are capable of forming a lytic pore in encapsulation vesicles can also be used. Other synthetic pore forming agents described in Regen et al, Biochem. Biophys. Res. Commun. 159:566-571, 1989, herein incorporated by reference.

The composition of the invention can also include fragments of naturally occurring or synthetic pore forming agents that exhibit lytic activity. In addition, the invention provides for biologically active and inactive fragments of polypeptides. Biologically active fragments are active if they are capable of forming one or more lesions or pores in synthetic or naturally occurring membrane systems. Inactive fragments are pore forming agents that are capable of being activated or cleaved into activity by some internal or external event, physical activity, or chemical modification.

The biologically active fragments of lytic pore forming agents can be generated by methods know to those skilled in the art such as proteolytic cleavage or recombinant plasmids.

The invention further comprises analogs of naturally occurring pore forming agents that may be capable of lysing cells. These analogs may differ from the naturally occurring pore forming agents by amino acid sequence differences or by modifications that do not affect sequence, or both.

Modifications include in vivo or in vitro chemical derivatization of polypeptides, e.g., acetylation, or carboxylation. Also included in the spirit of the invention are modifications of glycosylation and those made by modifying the glycosylation patterns of a polypeptide during its synthesis and processing steps.

The invention also comprises analogs in which one or more peptide bonds have been removed and replaced with an alternative type of bond or an alternative type of covalent bond such as a “peptide mimetic”. These mimetics are well known in the art. Similarly, the replacement of the L-amino acid residues is a standard way of rendering the polypeptide less sensitive to proteolysis. Also included are blocking groups that are used at the amino terminal end including: t-butyloxycarbonyl, acetyl, theyl, succinyl, methoxysuccinyl, suberyl, adipyl, azelayl, dansyl, benzyloxcarbonyl, fluorenylmethoxycarbonyl, methoxyazelayl, methoxyadipyl, methoxysuberyl, and 2,4 dinitrophenyl.

Although most modifications are designed to make most proteins more resistant to degradation, the present invention also comprises modifications that may be used to enhance such modifications or degradations.

Also within the scope of the present invention are naturally or synthetically occurring organic and inorganic molecules that may be combined with proteins or constructs of the present invention to make them less susceptible to immunological attack. For instance, the compound of the present invention may be coupled to molecules such as polyethylene glycol (PEG) or monomethoxy-polyethylene glycol (mPEG).

The invention comprises modifications that result in an inactive pore forming agent that can be activated by cell associated substances or conditions. Such modifications can include peptides containing enzymatic cleavage sites (lysine and arginine bonds that can be cleaved) or chemically reactive groups that can be photo-activated. Modifications also include peptides that may be modified to optimize solubility properties or to mediate activation by cell-associated substances.

The invention comprises peptides and genetic variants both natural and induced. Induced mutants can be made in a variety of methods known in the art including random mutangenesis or polymerase chain reaction.

The invention also comprises the use of organic and non-organic nanotubes. For instance, these molecules may comprise hollow coiled molecules, linear D,L peptides from cylindrical β or Π helices, helices folding of linear oligophenylacetylenes, ring stacking motifs, tubular ensembles form cyclic D,L-α-peptides, microcrystalline peptide nanotubes, self assembling transmembrane ion channels, pore structures from D,L peptides, cystine macrocycles, serinophanes, carbohydrate nanotubes, tubular mesophases from macrocyclic precursors, sector assembly motifs, nanotubes from block copolymers, folded sheet motifs, and others. For information regarding potential structures, structure design, construction and applicable materials and agents with the present invention, See “Self-Assembling Nanotubes”, M. R. Ghadiri et al., Angew. Chem. Int. Ed. 2001, 40, 988-1011; WO 95/10535 entitled “Cyclic peptide Tube”; WO 02/090503 A2 and early provisional application entitled “Antimicrobial Peptides and Compositions” (herein incorporated by reference in their entirety).

Solid-State Materials

The activation agents may also comprise solid-state materials that are both capable of being on the surface of an encapsulation vesicle or enclosed by the encapsulation vesicles. These materials generally are in inactive form. For instance, the activation agent may be or may comprise a nanoparticle, a magnetic nanoparticle, nanorod, a nanocomposite, a nanowire, a carbon nanotube, a nanodot, a quantum dot, a nanostructure, a nanoshell, materials comprising gold and/or silica and a nanomaterial. Magnetic nanoparticles may comprise probes with or without antibodies. Nanoparticles may also comprise various magnetic materials. Magnetic materials may provide for hyperthermia in defined tissues. For instance, the nanoparticles may comprise magnetic material such as ferrous oxide or ferric oxide. These magnetic materials may comprise a core. The core may be surrounded by a silica shell. An antibody or other targeting agent may be attached to the silica shell. This can be accomplished by way of a carbon spacer. Each of these materials must be capable of activation by an activation condition. Activation may include lytic activity, change or energy state by irradiation and/or degradation or release of materials that may prove toxic to a pathogenic cell. The activation agent may also comprise a combination or mixture of one or more of these agents. Activation may be chemical, by change in pH, by light or irradiation by UV, IR, microwave, heat or other physical or chemical agent to focus energy or change the material from a lower to higher energy state.

Targeting Ligand:

A targeting ligand may be optionally employed with the present invention. Targeting ligand refers to any material or substance that may promote targeting of tissues and/or receptors in vivo or in vitro with the compositions of the present invention. The targeting ligand may be optional employed with the present invention. A key property of the targeting ligand is the ability for the ligand to bind, attach or associate with the surface of a pathogenic cell.

The targeting ligand may be synthetic, semi-synthetic, or naturally occurring. Materials or substances that may serve as targeting ligands include, for example, proteins, antibodies, antibody fragments, hormones, hormone analogues, glycoproteins and lectins, peptides, polypeptides, amino acids, sugars, saccharides, including monosaccharides and polysaccharides, carbohydrates, vitamins, steriods, steriod analogs, hormones, cofactors, bioactive agents, genetic material, including nucleotides, nucleosides, nucleotide acid constructs and polynucleotides. The targeting ligands may include fusion proteins, monoclonal or polyclonal antibodies, Fv fragments, Fab′ or (Fab′)₂ or any similar reactive immunolgically derived component that may be used for targeting the constructs. Targeting ligands can also include other ligands, hormones, growth hormones, opiod peptides, insulin, epidermal growth factor, insulin like growth factor, tumor necrosis factors, cytokines, fibroblasts or fibroblast growth factors, interleukins, melanocyte stimulating hormone, receptors, viruses, cancer cells, immune cells, B cells, T-cells, CD4 or CD4 soluble fragments, lectins, concavalins, glycoproteins, molecules of hemopoetic origin, integrins and adhesion molecules. Other targeting ligands may be used in conjunction with the photodynamic pore forming agents. For instance, the seringe portion of the diphtheria toxin may be attached to a ligand and the constructs inserted into the encapsulation vesicles. These constructs could then be used to target or deliver the vesicles with the photodynamic pore forming agents. In addition, ligands may be employed to form immunoliposomes. For instance, ligands may comprise the anti-HER2 antibody that provides specific binding to the HER2-overexpressing cancer cells. Other immunoliposomes may be designed and coated with non-antibody ligands such as the folic acid/folate receptor, CKD 602 antibody/receptor, antintegrins and sialyl lewis (x)-oligosaccharide ligands attached to mPEG or PEG ligands extending from the liposome surface. These ligands once bound to the target molecule on the surface of tumor cells, result in uptake and internalization of the entire immunoliposome (including the encapsulated drug or activation agent). Once internalized immunoliposomes comprising the self-assembling activation agents can be activated to self assemble and destroy the cell or tumor or if already assembled to release a drug.

The Encapsulation Vesicles:

The encapsulation vesicle is important to the present invention and has a few important properties. The encapsulation vesicle must be capable of accommodating an activation agent inside its membrane. This may include the option of being able to attach a targeting ligand to the surface of the encapsulation vesicle. The encapsulation vesicle may also have the ability to encapsulate a bioactive compound. In addition, the encapsulation vesicle need not be a synthesized material. For instance, it may be naturally occurring or comprise parts of naturally occurring cells. For instance, the encapsulation vesicle may comprise a red blood cell, a white blood cell, a red blood cell ghost, a white blood cell ghost, a pathogenic cell, a diseased cell, or any other cell that has been infected or not infected. However, as discussed above, the encapsulation vesicle must be capable of associating or retaining one or more activation agents within its membrane. In other cases the encapsulation vesicles may be synthetic.

For instance in certain instances “vesicle” or “encapsulation vesicle” refers to an entity that is generally characterized by the presence of one or more walls or membranes that form one or more internal voids. Vesicles may be formulated, for example, from a stabilizing material such as a lipid, including the various lipids described herein, a proteinaceous material, including the various proteins described herein, and a polymeric material, including the various polymeric materials described herein. As discussed herein, vesicles may also be formulated from carbohydrates, surfactants, and other stabilizing materials, as desired. The lipids, proteins, polymers and/or other vesicle forming stabilizing materials, may be natural, synthetic or semi-synthetic. Preferred vesicles are those which comprise walls or membranes formulated from lipids. The walls or membranes may be concentric or otherwise. The stabilizing compounds may be in the form of one or more monolayers or bilayers. In the case of more than one monolayer or bilayer, the monolayers or bilayers may be concentric. Stabilizing compounds may be used to form a unilamellar vesicle (comprised of one monolayer or bilayer), an oligolamellar vesicle (comprised of more than about three monolayers or bilayers). The walls or membranes of vesicles may be substantially solid (uniform), or referred to as, for example, liposomes, lipospheres, Stealth® liposomes, nanoliposomes, particles, nanoparticles, micelles, bubbles, microbubbles, microspheres, nanospheres, fusogenic liposome, SNALP, SPLP (small plasmid lipid particles. Because the SPLP particles are small (˜100 nm), have a uniform size, low surface charge, and are stable and do not aggregate, they remain intact in circulation for many hours. These features of the SPLP and the SNALP allow the particles to accumulate at sites of vascular leak.), nanostructures, microballoons, microcapsules, aerogels, clathrate bound vesicles, hexagonal/cubic/hexagonal II phase structures, and the like. The internal void of the vesicle may be filled with a wide variety of materials including, for example, water, oil, gases, gaseous precursors, liquids, fluorinated compounds or liquids, liquid perfluorocarbons, liquid perfluoroethers, therapeutics, bioactive agents, if desired, and/or other materials. The vesicles may also comprise a targeting ligand if desired. The encapsulation vesicles may also include nanoerythrosomes and other lipid based or cellular derived materials. In addition, the vesicles may comprise parts of cell, other diseased or pathogenic cells capable of fusion or having receptors or fusion proteins on their surfaces. For instance, a potential encapsulation vesicle may comprise a virus such as a T4 phage, an adenovirus, a polio virus, an influenza virus, an HIV virus or other viruses, bacteria, fungi, or pathogenic cells capable of membrane fusion. These vesicles may be naturally occurring or may have been altered physically or chemically through recombinant DNA technology. However, other naturally occurring or non-naturally occurring synthetic and non-synthetic organic or biologically based molecules, polymers and co-polymers are within the scope of the invention. Naturally occurring encapsulation vesicles may include erythrocytes, leukocyte, melanocytes, fibroblasts or components of these cells. Other encapsulation vesicles may include synthetically designed organic molecules and biodegradable polymers are also within the scope of the present invention.

A vesicle may comprise a solid, substantially solid, gel, sol-gel, composite, nanocomposite, nanostructure, nanoporous material, porous nanostructure, nanoshell, nanocrystal, degradable polymer, biodegradable polymer, or device as taught in U.S. Pat. No. 3,948,254 (herein incorporated by reference). Other structures that are well known in the art include nanostructures that self-assemble. For instance such structures are described by Whitesides et al., Science (1991) 254: 1312-1319. Bates, Science (1991) 251: 898-905; Gunther & Stupp, Langmuir (2001) 17:6530-6539; Hulteen et al., J. Am. Chem. Soc. (1998) 120: 6603-6604; Moore and Stupp., J. Am. Chem. Soc. (1992): 9-14; Muthukumar et al, Science (1997) 277:1225-1232; Stupp et al., Science (1997) 276:384-389; Stupp et al., Science (1993) 259:59-63; and Zubarev et al., Science (1999) 283:523-526.

Activation Conditions or Substances:

An important component of the invention are the activation agents and the conditions to activate them. For instance, an activation agent such as a pore forming agent may be activated before or after release from the encapsulation vesicle. This may be accomplished by conditions or substances that are endogenously provided by the system or target cell or exogenously provided by a source other than the target cell. Physical, chemical or biochemical conditions may be used to activate the lytic activity. Physical conditions include heat, light or temperature changes. Chemical activators include changes in pH or reduction potential, metal ions or protecting groups that may be activated or de-activated. Light sources may include lasers, red lasers, infrared sources, ultraviolet lights, and other optical materials or substances well known in the art. In addition, activation may be accomplished by the insertion of a stent into a patient. The stent is inserted and then can be switched on or off to emit a light, UV or IR beam to activate the activation agent.

Assembly of the System:

The general assembly of the system is accomplished by techniques that will encapsulate the activation agents in the encapsulation vesicles. This has been done generally using active and passive systems. For instance, activation agents can be loaded into encapsulation vesicles at different stages, depending on their physical and chemical properties. For example, the activation agent can be included in the aqueous solution during hydration. In this method, referred to as passive loading, encapsulation vesicle formation and drug encapsulation occur at the same time. Alternatively, the drug can be loaded after the encapsulation vesicles are formed, a process called active loading. This strategy is used in the encapsulation of a number of modern drugs. In this case, the hydration step is performed to encapsulate an ammonium sulfate solution. After size reduction of the encapsulation vesicles, extraliposomal ammonium sulfate is removed by diafiltration. The activation agent is then added to the liposome preparation. The absence of the ammonium sulfate in the extra-encapsulation phase establishes a chemical gradient that induces the drug to diffuse into the encapsulation vesicle and become trapped inside. Active loading is usually more effective than passive loading. More than 90% of the added activation agent becomes encapsulated during the loading, while the typical efficiency of passive loading ranges from 20% to 40%. After loading, the unencapsulated drug can be removed by diafiltration or ionic exchange methods if needed. The preparation may then be sterilized by passage through a 0.2 micron sterilization membrane and filled into final product vials. When needed, the product can be lyophilized for added stability.

Components (i.e. monomers, cyclic peptides etc.) of the composition of the present invention are self-assembling. Under certain conditions they may be designed so that they do not self assemble in cell membranes or walls. For instance, the interior of the encapsulation vesicle may be raised to a range of from around 7.0 to around 14.5. This basic environment can then inactivate certain activation agents. A cyclic peptide or nanotube can be inactivated if it has amino acid residues with ionizable side chains. For instance, the cyclic peptides or nanotubes may comprise a number of glutamic acid residues that when deprotonated under basic conditions will not allow the units to self assemble to form supramolecular structures. When activated, the composition may be assembled in any order. Self-assembly may be molecular based where there is a spontaneous association of molecules under equilibrium conditions that form stable, structurally well defined aggregates joined by covalent or non-covalent bonds. For instance, if a pore forming agent such as a nanotube is employed, the nanotube may be delivered site specifically to a tumor or cancer cell and then allowed to self assemble. The nanotubes can be designed to self assemble in particular kinds of cell membranes or under particular environments or conditions. For instance the nanotubes or cyclic peptides can be protonated when they contact an acidic environment in an endosome. This will cause these activation agents to self assemble by hydrogen bonding into supramolecular structures in the nearest membranes. In addition, if the cyclic peptides or nanotubes have been previously screened or selected for a particular cell type, they will mostly likely assemble only in these types of cells.

Therapeutic Composition:

The therapeutic composition comprises an encapsulation vesicle for encapsulating a bioactive agent and an activation agent such as a self assembling nanotube enclosed in the encapsulation vesicle.

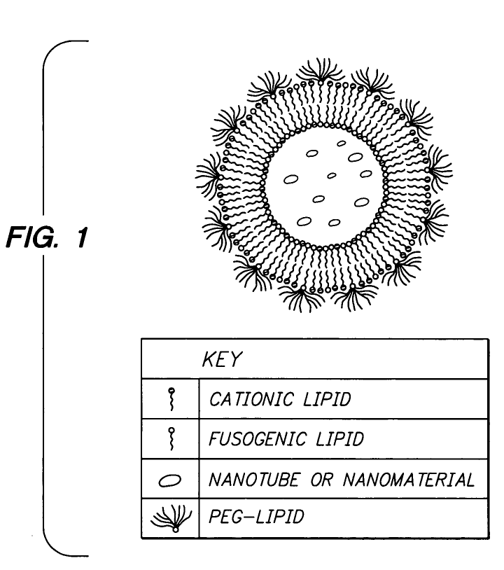

FIG. 1 shows a schematic view of a first embodiment of the present invention. The figure shows an encapsulation vesicle such as a fusogenic liposome and an activation agent such as an organic nanotube enclosed inside the membrane of the encapsulation vesicle. In the figure the activation agent may comprise a nanotube, cyclic peptide, or D,L-α-peptide or similar type molecule. The nanotube may be designed to be activated to self assemble by an activation condition. In addition, the molecules may self assemble upon release to cells particularly susceptible to the activation agent.

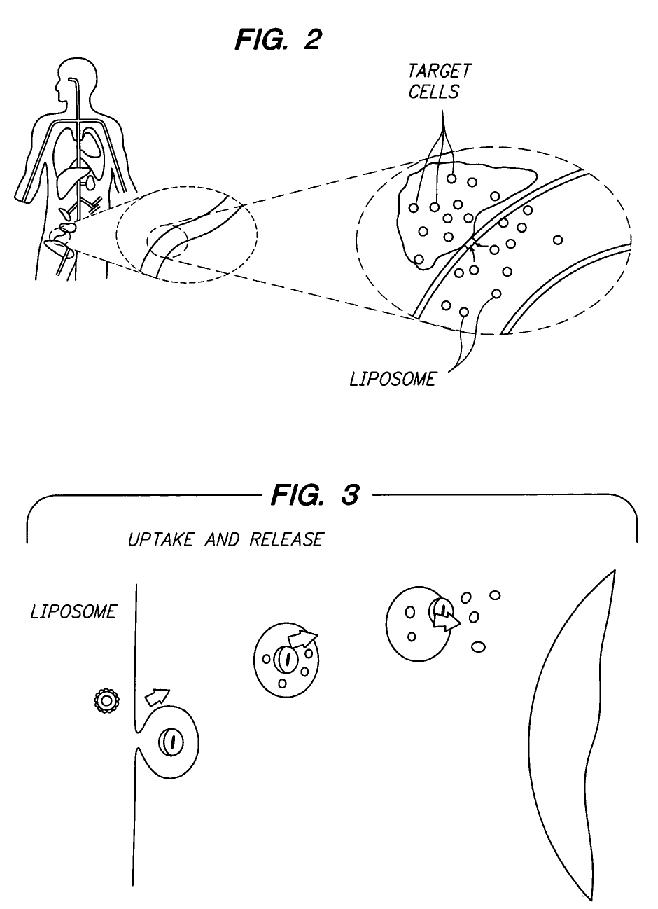

FIG. 2 shows the therapeutic composition and how it may be employed. The therapeutic composition circulates in the blood stream and is extravasated by a tissue or tumor. The activation agents can then be activated and released inside the tumor cells to self assemble in their membranes. Activation agents can directly self assemble or can be derivatized or modified so that an activation condition can be employed to activate them to self assemble.

FIG. 3 shows how the therapeutic composition is taken up by a particular type of cell. This may be by phagocytosis, endocytosis, pinocytosis, endocytosis with chathrate and coated pits, transfection, fusion, etc. The invention works with all modes of entry, but is shown with and endocytosis process. Once taken up, the endosomes fuses with a lysosome and the activation agent is released and activated.

Therapeutic Administration:

The composition can be administered to and methods performed on an animal or human suffering from a medical disorder or disease. The composition may be used alone or in combination with other chemotherapeutic or cytotoxic agents. The encapsulation vesicles can contain a bioactive agent used to treat a disease. For example an oligomeric antisense DNA or nucleic acid could be used in the carrier for delivery to a diseased or pathogenic cell. Other bioactive agents used for treating cancer and HIV could also be used.

The composition may also be administered and methods performed by intravenous infusion, subcutaneous injection, or direct injection to the site of infection by a stent, laparoscope or other similar type device. The present invention could also be applied topically or aspirated to a tumor site via bronchial passages to treat cancers of the lung. In addition, the administration may be by oral, nasal, parenteral (including subcutaneous, intravenous, intramuscular, and intraperitoneal), rectal, dermal, transdermal, intrathoracic, intrapulmonary, intranasal (respiratory routes). The therapeutic may also be formulated for sustained release (microencapsulation, See WO 94/07529).

### EXAMPLE 1

Activation agents:

Construction of the activation agents has been previously discussed. For instance, activation agents have been discussed in WO 2003092632 A2 entitled “Cyclic Peptide Anticancer Agents and Methods”, Filed Jun. 5, 2003; WO 2003092631 A2 entitled “Cyclic Peptide Anti-Viral Agents and Methods”, Filed Jun. 5, 2003; WO 2003093300 A2, Entitled “Anti-Microbial Peptides and Compositions”, Filed Jun. 5, 2003; and EPO Patent No. EPO 723554 B1 (these references are herein incorporated by reference in their entirety). Although a variety of activation agent may be employed, the present invention may find cyclic D,L-α-peptides or organic nanotube particularly useful. In particular, the cyclic peptides can be selected for treating a particular type of cell such as a cancer cell. The present invention particularly works well using peptides selected for use and application to cancer cells. These activation agents can then be further screened to determine the best candidates for use or inactivation by a specific activation condition. For instance, selection of activation agents high in amino acids with ionizable side chains such as glutamic acid, aspartic acid, tyrosine, tryptophan, ornithine, and cystine are useful under basic pH conditions to inactivate the activation agents. These same activation agents will then become active when protonated under acid conditions.

### Example 2

Cell and Condition Specific Activation Agents:

Various activation agents may be selected by testing in vitro activity. This is not a requirement of the invention. For instance, Lopez et al., “Antibacterial agents based on the cyclic D,L-α-peptide architecture, Letters to Nature, Volume 412, Jul. 26, 2001, describes a methodology for selection of particular cyclic peptides that self assemble in bacterial membranes as opposed to mammalian cell membranes. Selectivity is based on substitution of various amino acids in the cyclic peptides. Similar screening activities can be conducted for other types of bacteria and diseased cells. For instance, screening activity has been done for cancer cells, viral cells, and bacterial cells. See WO 092632 A2 entitled “Cyclic Peptide Anticancer Agents and Methods”, filed Jun. 5, 2003; WO 092631 A2 entitled “Cyclic Peptide Anti-Viral Agents and Methods”, filed Jun. 5, 2003; and WO 093300 A2, Entitled “Anti-Microbial Peptides and Compositions”, filed Jun. 5, 2003.

The present invention uses a particular type of activation agent that is capable of activation when exposed to proper pH. For instance, the kinetics of assembly and disassembly of cyclic peptides to form molecular tubes can be controlled by the selection of amino acid side chain groups. Cyclic peptide with ionizable amino acid side chains display pH dependent kinetics. Charged cyclic peptides are found to resist tube assembly; neutralized cyclic peptides are found to promote self assembly. For example, as further described in EPO Patent No. EPO 723554 B1 (see Example 1), experiments showing cyclic peptides incorporating glutamic acid and other similar charged amino acids were found to spontaneously assemble into molecular tubes at acidic pH, but resist assembly into molecular tubes at alkaline pH. Pre-assembled molecular tubes were found to spontaneously disassemble when the pH was raised from acid pH to alkaline pH. Judicious selection of amino acid side chains can also promote packing or aggregation of molecular tubes to form molecular bundles. Molecular bundles then will not self assemble in the encapsulation vesicle that encloses them. This allows for drug loading of the molecular bundles that may be encapsulated by the encapsulation vesicles.

### Example 3

Encapsulation Vesicles:

The use of an encapsulation vesicle with an activation agent such as a cyclic D,L-α-peptides have a number of problems because cyclic D,L-α-peptides are capable of self assembly in their membranes. However, as discussed, the internal environment of the encapsulation vesicle can be altered so that the pH on the inside of the encapsulation vesicle is more basic. For instance, the pH of the internal environment can be raised to above pH 7.5 (for example, somewhere between 7.5 and 10.5 pH). This will cause the activation agents to deprotonate and not self assemble to form supramolecular structures in the encapsulation vesicles. A number of encapsulation vesicles can be altered for use in this invention. For instance, regular liposomes and various fusogenic liposomes may be employed. Other encapsulation vesicles may comprise liposome complexes as taught in U.S. Pat. No. 6,372,720 B1, entitled “Liposome Fusion and Delivery Vehicle”, issued on Apr. 16, 2002; liposomes as taught in U.S. Pat. No. 5,013,556, Entitled “Liposomes with Enhanced Circulation Time”, issued on May 7, 1991; pH sensitive liposomes as discussed in U.S. Pat. No. 5,595,756, entitled “Liposomal Compositions for Enhanced Retention of Bioactive Agents”, issued on Januar 21, 1997; liposomes as taught in U.S. Pat. No. 6,417,326, entitled “Fusogenic Liposomes”, issued on Jul. 9, 2002; and Liposomes and components taught in U.S. Pat. No. 5,885,613, entitled “Bilayer Stabilizing Components and Their Use in Forming Programmable Fusogenic Liposomes”, issued on Mar. 23, 1999. Each of these patents and applications is herein incorporated by reference in its entirety.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989)). When liposomes are endocytosed by a target cell, for example, they can be routed to acidic endosomes which will destabilize the liposome and result in drug release. FIG. 1 shows the fusogenic liposome that may be employed with the present invention. The liposome comprises cationic lipids, PEG-lipids, an activation agent such as a nanomaterial or nanotube and a fusogenic lipid. In certain instances the nanotube will comprise a nanotube or cyclic peptide having one or more amino acids with ionizable side chains. The liposome can be intravenously administered as shown in FIG. 2. The fusogenic liposome with enclosed organic nanotube or cyclic peptide then circulates through the patient's blood stream to a vessel structure or tumor. The fusogenic liposome then extravasates into the targeted cells.

Following accumulation at the target site, the liposomal carrier becomes fusogenic, without the need for any external stimulus, and subsequently releases any encapsulated or associated drug or therapeutic agent in the vicinity of the target cell, or fuses with the target cell plasma membrane introducing the drug or therapeutic agent into the cell cytoplasm. In certain instances, fusion of the liposomal carrier with the plasma membrane would be preferred because this would provide more specific drug delivery and, hence, minimize any adverse effects on normal, healthy cells or tissues. In addition, in the case of therapeutic agents such as DNA, RNA, proteins, peptides, etc., which are generally not permeable to the cell membrane, such a fusogenic carrier would provide a mechanism whereby the therapeutic agent could be delivered to its required intracellular site of action.

FIG. 3 shows the present invention provides a fusogenic liposome comprising a lipid capable of adopting a non-lamellar phase, yet capable of assuming a bilayer structure in the presence of a bilayer stabilizing component; and a bilayer stabilizing component reversibly associated with the lipid to stabilize the lipid in a bilayer structure. The encapsulation vesicle may also comprise a lipopeptide or liponanotube that essentially comprises a lipid covalently attached to an organic nanotube or cyclic peptide by means of an amide bond. Typically, the amide bond is formed between a carboxyl group of the lipid and an amino group of the organic nanotube or cyclic peptide.

In FIG. 3, the fusogenic liposome carrying the activation agent such as the cyclic peptide or organic nanotube is delivered close to the cancer cells by extravasation. Once in the vicinity the fusogenic liposome is taken up by endocytosis, fusion, or receptor mediated endocytosis. During endocytosis the cell's membrane surrounds the liposomes, wrapping it in an envelope of membrane lipids that are similar to those of the liposome. This envelope or endosome pinches off from the cell's membrane and migrates to the inside of the cell. The internalized liposome undergoes an interaction with the endosomal membrane which causes a lowering of the pH. This causes both a collapse and/or release of the organic nanotube or cyclic peptides from the endosome into the cytoplasm. In addition, the lowering of the pH causes protonation or activation of the cyclic peptides. The cyclic peptides or organic nanotubes will then self assemble into supramolecular structures in the closest membranes. In other words, they self assemble in the lipid bilayers of the cancer cells and destroy them. It is within scope of the invention that variations in pH may be employed such as an acidic environment etc. In addition, it is also within the scope of the invention that other activation conditions may be employed to change the activation agent from an active or inactive state to a similar or different state. Other activation conditions as described above may be employed.

The invention having been thus described, what is claimed as new and desired to secure as Letters Patent is:

1. A liposome for introducing an organic nanotube into a cell, comprising:
(a) a lipopeptide, said lipopeptide including a lipid covalently attached to a peptide; and
(b) an inactivated organic nanotube enclosed in the liposome.

2. A liposome according to claim 1, wherein the inactivated organic nanotube comprises a cyclic peptide.

3. A liposome according to claim 2, wherein the cyclic peptide comprises a cyclic D,L-α-peptide.

4. A liposome according to claim 3, wherein the cyclic D,L-α-peptide comprises from 1 to 38 amino acids.

5. A liposome according to claim 1, wherein the inactivated organic nanotube is activatable.

6. A liposome according to claim 5, wherein the inactivated organic nanotube is activatable by a change in pH.

7. A liposome according to claim 5, wherein the inactivated organic nanotube is activatable by a chemical reaction.

8. A liposome according to claim 5, wherein the inactivated organic nanotube is activatable by a change in temperature.

9. A liposome according to claim 5, wherein the inactivated organic nanotube is activatable by electromagnetic radiation.

10. A liposome according to claim 9, wherein the electromagnetic radiation is selected from the group consisting of ultraviolet radiation, visible radiation, infrared radiation and microwave radiation.

11. A liposome according to claim 1, wherein the inactivated organic nanotube is from 1 to 38 amino acids.

12. A liposome according to claim 1, wherein the liposome comprises an interior that has a pH from about 7.0 to about 14.5.

13. A method according to claim 1, wherein the liposome comprises an interior that is maintained at a pH from about 7.0 to about 10.5.

14. A liposome according to claim 1, wherein the lipid is attached to the peptide by way of an amide bond.

15. A liposome according to claim 1, wherein the lipopeptide comprises a fusion peptide.

16. A liposome according to claim 1, wherein the lipopeptide comprises a lysine residue at the C-terminus.

17. A liposome according to claim 2, wherein the cyclic peptide comprises one or more glutamic acid residues.

18. A liposome according to claim 2, wherein the cyclic peptide comprises one or more amino acids having at least one ionizable side chain.

19. A liposome according to claim 4, wherein the cyclic D,L α peptide comprises one or more ionizable amino acids that when exposed to a pH which allows protonation sufficient to cause self assembly of the cyclic D,L α peptide will cause the cyclic D,L α peptide to self assemble into a supramolecular structure.

20. A liposome according to claim 19, wherein the pH is below about 7.0.

21. A liposome according to claim 4, wherein the cyclic D,L α peptide comprises an amino acid having at least one ionizable side chain, the amino acid selected from the group consisting of aspartic acid, glutamic acid, lysine, arginine, tyrosine, ornithine, histidine, serine, and cystine.

22. A pharmaceutical composition for introducing an organic nanotube into a cell of a host, comprising:
(a) a liposome, the liposome comprising a lipopeptide, said lipopeptide comprising a lipid covalently attached to a peptide;
(b) an inactivated organic nanotube enclosed in the liposome; and
(c) a pharmaceutically acceptable carrier.

23. A method of introducing an organic nanotube into a cell, comprising:
(a) contacting a cell with a composition comprising a liposome comprising
i. a lipopeptide, said lipopeptide including a lipid covalent attached to a peptide, and
ii. an inactivated organic nanotube enclosed in the liposome; and
(b) allowing the cell to take up the composition.

24. A method according to claim 23, wherein the lipopeptide comprises a fusion peptide.

25. A method according to claim 23, wherein the inactivated organic nanotube comprises a cyclic peptide.

26. A method according to claim 25, wherein the cyclic peptide comprises a cyclic D,L-α-peptide.

27. A method according to claim 23, wherein the liposome comprises an interior that is maintained at a pH from about 7.0 to about 14.5.

28. A method according to claim 23, wherein the liposome comprises an interior that is maintained at a pH from about 7.0 to about 10.5.

29. A method according to claim 23, wherein the inactivated organic nanotube is activated by a change in pH.

30. A method according to claim 23, wherein the inactivated organic nanotube is activated by a chemical reaction.

31. A method according to claim 23, wherein the inactivated organic nanotube is activated by a change in temperature.

32. A method according to claim 23, wherein the inactivated organic nanotube is activated by electromagnetic radiation.

33. A method according to claim 32, wherein the electromagnetic radiation is selected from the group consisting of ultraviolet radiation, visible radiation, infrared radiation, and microwave radiation.

34. A method of treating a patient comprising:
(a) introducing into a patient a first pharmaceutical composition, comprising a liposome comprising
i. a lipopeptide, said lipopeptide including a lipid covalent attached to a peptide, and
ii. an inactivated organic nanotube enclosed in the liposome; and
(b) introducing into the patient a pharmaceutically effective amount of a second pharmaceutically active composition, or a pharmaceutically acceptable salt thereof, whereby the first pharmaceutical composition and the second pharmaceutical composition are present in the patient simultaneously.

35. A method according to claim 34, wherein the lipopeptide comprises a fusion peptide.

36. A method according to claim 34, wherein the inactive organic nanotube comprises a cyclic peptide.

37. A method according to claim 36, wherein the cyclic peptide comprises a cyclic D,L-α-peptide.

38. A method according to claim 34, wherein the liposome comprises an interior that is maintained at a pH from about 7.0 to about 14.5.

39. A method according to claim 34, wherein the liposome comprises an interior that is maintained at a pH from about 7.0 to about 10.5.

40. A method according to claim 34, wherein the inactivated organic nanotube is activated by a chemical reaction.

41. A method according to claim 34, wherein the inactivated organic nanotube is activated by a change in pH.

42. A method according to claim 34, wherein the inactivated organic nanotube is activated by electromagnetic radiation.

43. A method according to claim 42, wherein the electromagnetic radiation is selected from the group consisting of ultraviolet radiation, visible radiation, infrared radiation, and microwave radiation.

44. A method according to claim 34, wherein the inactivated organic nanotube is activated by a change in temperature.

45. A method according to claim 34, wherein the inactivated organic nanotube is activated by a chemical reaction.

46. A method according to claim 34, wherein the second pharmaceutically active composition comprises an antitumor composition.

47. A method according to claim 34, wherein the second pharmaceutically active composition comprises an antiviral composition.

48. A method according to claim 34, wherein the second pharmaceutically active composition comprises an antibacterial composition.
